(19) United States
(12) Patent Application Publication
(10) Pub. No.: US 2022/0330912 A1
(43) Pub. Date: Oct. 20, 2022

(54) METHOD, SYSTEM, AND NON-TRANSITORY COMPUTER-READABLE RECORDING MEDIA FOR ANALYZING BREATHING-RELATED SOUND

(71) Applicant: DAIN TECHNOLOGY, INC., Seoul (KR)

(72) Inventors: Jee Young SONG, Seoul (KR); Ji Young JUNG, Gyeonggi-do (KR)

(21) Appl. No.: 17/760,856

(22) PCT Filed: Sep. 17, 2020
(86) PCT No.: PCT/KR2020/012567

§ 371 Date: Mar. 16, 2022

(30) Foreign Application Priority Data
Sep. 17, 2019 (KR) 10-2019-0113926

(51) Int. Cl. A61B 7/00; A61B 5/00; A61B 7/04

(52) U.S. Cl. CPC A61B 7/003; A61B 5/725; A61B 5/7203; A61B 5/7267; A61B 5/7257; A61B 7/04

(57) ABSTRACT

An aspect of the present invention provides a method for analyzing a breathing-related sound, the method comprising the steps of: receiving a sound signal obtained while a recording device is in contact with a body; filtering noises at least including a signal related to a heart sound from the sound signal; decomposing the filtered sound signal into a plurality of base signals based on empirical mode decomposition, wherein a first base signal has different frequencies from a second base signal; determining a base signal associated with a lung sound from the plurality of base signals; and obtaining lung-related information using a sound analysis model and the determined base signal.

### CROSS-REFERENCE TO RELATED APPLICATIONS

This application is a National Phase Entry of PCT International Application No. PCT/KR2020/012567, filed Sep. 17, 2020, and claims priority to Korean Patent Application No. 10-2019-0113926, filed Sep. 17, 2019, the entire contents of which are hereby expressly incorporated herein by reference.

### TECHNICAL FIELD

The present inventive concepts relate to method, system, and non-transitory computer-readable recording media for analyzing a breathing-related sound.

### BACKGROUND

Auscultation is one of the most basic traditional diagnostic methods to check the state of the circulatory and respiratory systems such as the heart and lungs during medical treatment. An experienced doctor can diagnose abnormalities and diseases of the heart and lungs through the sound heard during auscultation. In addition, such a diagnostic method is cost-effective because it is performed only with a stethoscope, and is still widely used to this day because it is not restricted by time and place.

However, there are disadvantages in that it takes a lot of time to perform auscultation beyond a certain level of proficiency because the proficiency is increased through training, and there are differences between medical staff and time series data could not be compared and evaluated in a consistent and quantitative way because the process of listening and judging is very subtle and subjective. In addition, even in the case of experienced doctors, there was a big limitation in evaluating whether the condition of the revisiting patient is improved or deteriorated compared to the previous condition.

Meanwhile, various sounds are generated in the body during breathing. In particular, a sound that does not have a specific frequency feature (e.g., white noise) is generated by normal person in the case of lung sounds, while it is known that a musical sound is generated due to a deformation in the shape of the organs through which air travels when an abnormality occurs in the lungs. That is, since the human respiratory system makes sounds similar to those of wind instruments, it is possible to detect or diagnose symptoms and diseases by auscultating the sounds.

The present inventor(s) has proposed a new and advanced technology that extracts the base signal associated with lung sounds from the sound signal generated from the body on the basis of empirical mode decomposition, and performs at least one of abnormal lung sound detection, respiratory disease diagnosis, and disease condition prediction in a quantitative, consistent, and accurate manner, on the basis of the abnormal lung sound analysis model that is learned using data on the features of the base signal.

### SUMMARY

An objective of the present inventive concepts is to solve the above problems described above in the related art.

Another objective of the present inventive concepts is to precisely analyze even minute amplitude lung sounds by analyzing sound signals generated from the body on the basis of empirical mode decomposition.

Another objective of the present inventive concepts is to determine a base signal associated with a lung sound from sound signals generated from the body and to perform learning on the basis of the base signal.

Another objective of the present inventive concepts is to quickly and accurately perform abnormal lung sound detection, respiratory disease diagnosis, and disease condition prediction on the basis of the sound generated from the body of the patient, without relying on an experienced physician.

A representative configuration of the present inventive concepts for achieving the above objectives is as follows.

According to an aspect of the present inventive concepts, a method of analyzing a breathing-related sound is provided, the method including acquiring a sound signal generated from the body; filtering out noises from the sound signal; decomposing the filtered sound signal into a plurality of base signals having different frequencies on the basis of empirical mode decomposition, and determining a base signal associated with a lung sound from the plurality of base signals; and enabling an abnormal lung sound analysis model to be learned using, as learning data, data on at least one feature specified from the base signal associated with the lung sound.

According to another aspect of the present inventive concepts, a system for analyzing a breathing-related sound is provided, the system including a sound signal acquisition unit acquiring a sound signal generated from the body; a filtering management unit filtering out noises from the sound signal; a base signal determination unit decomposing the filtered sound signal into a plurality of base signals having different frequencies on the basis of empirical mode decomposition, and determining a base signal associated with a lung sound from the plurality of base signals; and a leaning management unit enabling an abnormal lung sound analysis model to be learned using, as learning data, data on at least one feature specified from the base signal associated with the lung sound.

In addition to this, another method and system for implementing the present inventive concepts, and a non-transitory computer-readable recording medium for recording a computer program for executing the method are further provided.

According to the present inventive concepts, it is possible to precisely analyze even minute amplitude lung sounds by analyzing sound signals generated from the body on the basis of empirical mode decomposition.

In addition, according to the present inventive concepts, it is possible to determine a base signal associated with a lung sound from sound signals generated from the body and to perform learning on the basis of the base signal.

In addition, according to the present inventive concepts, it is possible to quickly and accurately perform abnormal lung sound detection, respiratory disease diagnosis, and disease condition prediction on the basis of the sound generated from the body of the patient, without relying on an experienced physician.

### BRIEF DESCRIPTION OF DRAWINGS

FIG. 1 is a diagram schematically showing the configuration of an entire system for analyzing a breathing-related sound according to the present inventive concepts.

FIG. 2 is a diagram illustrating an internal configuration of a sound analyzing system shown in FIG. 1 according to an embodiment of the present inventive concepts.

FIGS. 3 and 4 are diagrams illustrating a plurality of base signals according to an embodiment of the present inventive concepts.

FIGS. 5 to 17 are diagrams illustrating features of each symptom appearing in a base signal associated with a lung sound according to an embodiment of the present inventive concepts.

FIG. 18 is a diagram illustrating a process of analyzing a breathing-related sound according to an embodiment of the present inventive concepts.

### DETAILED DESCRIPTION

Detailed description of the present inventive concepts will refer to the accompanying drawings, which show, by means of illustration, example embodiments in which the present inventive concepts may be practiced. These embodiments are described in sufficient detail to enable those skilled in the art to practice the present inventive concepts. It should be understood that the various embodiments of the present inventive concepts are different but need not be mutually exclusive. For example, certain shapes, structures, and features described herein may be implemented in other embodiments with respect to one embodiment without departing from the spirit and scope of the inventive concepts. In addition, it should be understood that the location or arrangement of individual components within each of the disclosed embodiments may be changed without departing from the spirit and scope of the present inventive concepts. Accordingly, the detailed description given below is not intended to be taken in a limiting sense, but the scope of the present inventive concepts, if properly described, is limited only by the appended claims, along with all scope equivalents to those claimed. Like reference numerals in the drawings refer to the same or similar functions throughout the various aspects.

Hereinafter, in order to enable those of ordinary skill in the art to easily practice the present inventive concepts, embodiments of the present inventive concepts will be described in detail with reference to the accompanying drawings.

Configuration of the Whole System

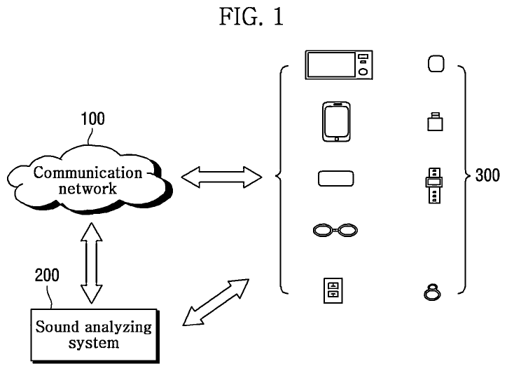

FIG. 1 is a diagram schematically showing the configuration of a whole system for analyzing a breathing-related sound according to the present inventive concepts.

As shown in FIG. 1, the whole system according to an embodiment of the present inventive concepts may include a communication network 100, a sound analyzing system 200, and a device 300.

First, the communication network 100 according to an example embodiment of the present inventive concepts may be configured with wired communication or wireless communication networks, such as a local area network (LAN), a metropolitan area network (MAN), a wide area network (WAN), and the like, regardless of a communication mode. Preferably, the communication network 100 as used herein may include a known local area wireless communication network, such as Wi-Fi, Wi-Fi Direct, LTE Direct, and Bluetooth. However, the communication network 100 may include, but not limited thereto, a known wired/wireless data communication network, a known telephone network, or a known wired/wireless television communication network.

For example, the communication network 100 may be configured by implementing, as a wireless data communication network, at least a portion of a traditional communication method, such as Wi-Fi communication, WiFi-Direct communication, Long-Term Evolution (LTE) communication, Bluetooth communication (including Bluetooth Low Energy (BLE)), infrared communication, ultrasonic communication, and the like. As another example, the communication network 100 may be configured by implementing at least a portion of an existing communication method such as LiFi (Light Fidelity).

Next, the sound analyzing system 200 according to an example embodiment of the present inventive concepts may perform steps of acquiring a sound signal generated from the body, filtering out noise from the sound signal, decomposing the filtered sound signal into a plurality of base signals having different frequencies on the basis of empirical mode decomposition, determining a base signal associated with a lung sound from the plurality of base signals, and enabling an abnormal lung sound analysis model to be learned using, as learning data, data on at least one feature specified from the base signal associated with the lung sound.

In addition, the sound analyzing system 200 according to an example embodiment of the present inventive concepts performs functions of performing learning on the abnormal lung sound analysis model and then performing at least one of abnormal lung sound detection, respiratory disease diagnosis, and disease condition prediction using the abnormal lung sound analysis model.

The function of the sound analyzing system 200 will be described in more detail below. Although the sound analyzing system 200 has been described above, it is apparent to those skilled in the art that this description is exemplary, and at least a part of functions or components required for the sound analyzing system 200 may be realized in the device 300 or included in the device 300 as necessary.

Finally, the device 300 according to an embodiment of the present inventive concepts is a digital device including a function of performing communication via access to the sound analyzing system 200, and any digital device equipped with a memory and a microprocessor to have arithmetic capability may be adopted as the device 300 according to the present inventive concepts. The device 300 may be a wearable device such as smart glasses, smart watch, smart band, smart ring, smart necklace, or the like, or a conventional device such as a smartphone, smart pad, desktop computer, notebook computer, workstation, PDA, web pad, mobile phone, and the like.

In particular, the device 300 according to an embodiment of the present inventive concepts may include a sensing module (e.g., a microphone, a sound collector, etc.) for acquiring a sound signal generated from the body, or may be connected to the sensing module via a communication network.

In addition, according to an embodiment of the present inventive concepts, the device 300 may further include an application for performing functions according to the present inventive concepts. Such an application may exist in the form of a program module in the device 300. The program module may be generally similar to a sound signal acquisition unit 210, a filtering management unit 220, a base signal determination unit 230, a learning management unit 240, an analysis management unit 250, a communication unit 260, and a control unit 270 in the sound analyzing system 200, as described later. Here, at least a part of the application may be replaced with a hardware device or a firmware device capable of performing substantially the same or equivalent function as the application, if necessary.

Configuration of Sound Analyzing System

Hereinafter, a configuration of the sound analyzing system 200 that takes an important role in the implementation of the present inventive concepts and a function of each component will be described.

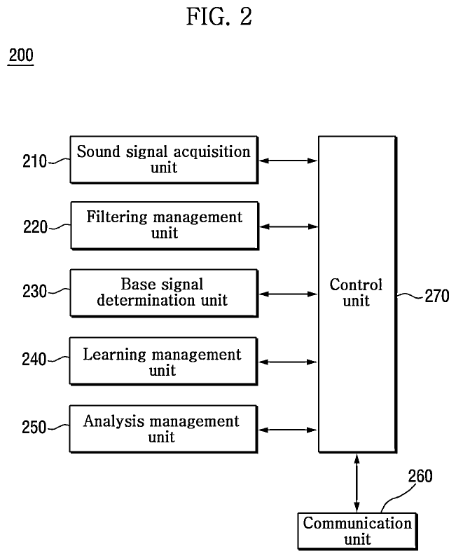

FIG. 2 is a diagram illustrating a configuration of a sound analyzing system 200 according to an example embodiment of the present inventive concepts.

Referring to FIG. 2, the sound analyzing system 200 according to an embodiment of the present inventive concepts may include a sound signal acquisition unit 210, a filtering management unit 220, a base signal determination unit 230, a learning management unit 240, an analysis management unit 250, a communication unit 260, and a control unit 270. According to one embodiment of the present inventive concepts, at least some of the sound signal acquisition unit 210, the filtering management unit 220, the base signal determination unit 230, the learning management unit 240, the analysis management unit 250, the communication unit 260, and the control unit 270 in the sound analyzing system 200 may be program modules that communicate with an external system (not shown). These program modules may be included in the sound analyzing system 200 in the form of an operating system, an application program module, and other program modules, and may be physically stored in various known storage devices. In addition, these program modules may be stored in a remote storage device capable of communicating with the sound analyzing system 200. The program modules include, but are not limited to, routines, subroutines, programs, objects, components, data structures, and the like, which perform specific tasks or execute specific abstract data types, according to the present inventive concepts.

First, the sound signal acquisition unit 210 according to an embodiment of the present inventive concepts may perform a function of acquiring a sound signal generated from the body.

For example, according to an embodiment of the present inventive concepts, the user's smartphone 300 or a sensing module (e.g., a sound collector, a patch, etc.) connected with the smartphone 300 via the communication network 100 may be in contact with the predefined body part of the user (e.g., the part such as the airway, neck, chest, lung-liver boundary, etc.), and the sound signal acquisition unit 210 may acquire a sound signal generated from the contacted body in real time.

As another example, according to an embodiment of the present inventive concepts, a sound signal generated from the predefined body part of the user (e.g., a part of the airway, neck, chest, lung-liver boundary, etc.) is recorded through a stethoscope (for example, through a separate recording device or a user device 300 connected to the stethoscope), so that the sound signal acquisition unit 210 may acquire the recorded sound signal.

Next, the filtering management unit 220 according to an embodiment of the present inventive concepts may perform a function of filtering out noise from the sound signal obtained by the sound signal acquisition unit 210. The noise according to an embodiment of the present inventive concepts may include ambient background noises (e.g., human voice), impulse noises, heart sounds, and the like.

For example, the filtering management unit 220 may filter out noises from the sound signal using a window or a bandpass filter (or Butterworth filter) set on the basis of features of the noise. More specifically, the filtering management unit 220 may remove the heart sound from the sound signal by using a bandpass filter (or an 8th-order Butterworth filter) with pass band frequency from 20 Hz or more to 300 Hz or less, on the basis of the features of the heart sound.

For another example, the filtering management unit 220 may track signals corresponding to noise (for example, by utilizing a profile for each noise) by analyzing features related to the amplitude and frequency of the sound signal, and may adaptively filter the noise.

Meanwhile, the filtering management unit 220 may be configured to perform detrending for the sound signal obtained by the sound signal acquisition unit 210.

For example, the filtering management unit 220 may remove a linear or trend component of the sound signal by applying a predetermined moving average filter to the sound signal. Meanwhile, the detrending method according to the present inventive concepts is not necessarily limited to the above-listed methods, but may be variously changed within the scope capable of achieving the object of the present inventive concepts.

Next, the base signal determination unit 230 according to an embodiment of the present inventive concepts may perform functions of decomposing the sound signal filtered by the filtering management unit 220 into a plurality of base signals having different frequencies on the basis of empirical mode decomposition, and determining a base signal associated with a lung sound from the plurality of base signals. Here, the base signal may refer to a signal that satisfies the conditions that the number of local extrema and zero crossings in the whole signal is the same or differs by at most one, and the sum of average values of the envelopes of local maxima and local minima is 0. Such a signal may be specified on the basis of an intrinsic mode function (IMF) calculated by an empirical mode decomposition method. Meanwhile, the method of decomposing a certain signal into a plurality of base signals on the basis of the empirical mode decomposition according to an embodiment of the present inventive concepts may be performed with reference to the methods disclosed in Korean Patent No. 10-1646981 and Korean Patent No. 10-1539396 (which should be considered to be incorporated by reference herein in its entirety).

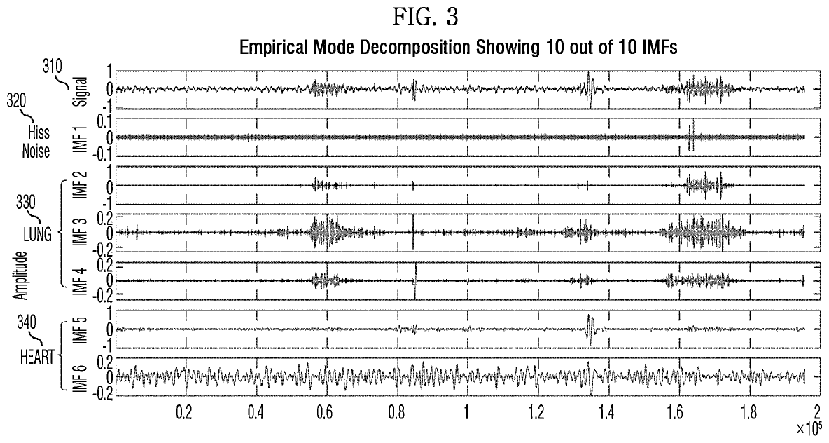

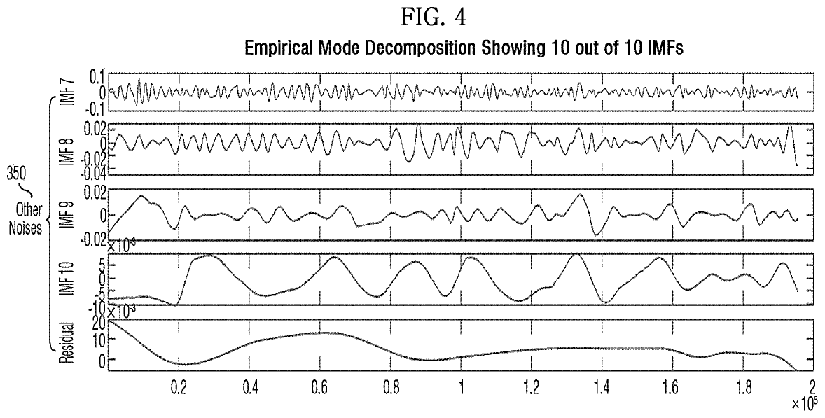

For example, referring to FIGS. 3 and 4, the base signal determination unit 230 may decompose the filtered sound signal 310 into a plurality of base signals 320, 330, 340, 350 having different frequencies (e.g., a plurality of intrinsic mode functions) on the basis of the empirical mode decomposition (specifically, it is decomposed into ten base signals and residual signals). Thereafter, the base signal determination unit 230 may determine a plurality of base signals 330 associated with the lung sound from among the 10 base signals 320, 330, 340, and 350. According to an embodiment of the present inventive concepts, the base signal determination unit 230 determines, as the base signal associated with the lung sound, a base signal of a specific sequence determined from among the plurality of base signals on the basis of the number of base signals. For example, when the sound signal is decomposed into ten base signals, the base signals corresponding to the second to the fourth high frequency in the order of the magnitude of the frequency may be preset as the base signal associated with the lung sound, and may be determined on the basis of a lookup table regarding this correspondence. In addition, the base signal determination unit 230 determines the base signal associated with the lung sound from among the plurality of base signals by referring to at least one of the amplitude, frequency feature, and waveform pattern of each base signal. For example, a base signal which is similar to the base signal associated with the lung sound by a certain level or more in at least one of amplitude, frequency features, and wave form is determined as the base signal associated with the lung sound, and a remaining base signal after removing a base signal which is less than the base signal associated with the lung sound by a certain level or more in amplitude, frequency features, and wave form is determined as the base signal associated with the lung sound.

Next, the learning management unit 240 according to an embodiment of the present inventive concepts may perform a function of learning an abnormal lung sound analysis model using, as learning data, data on at least one feature specified from the base signal associated with the lung sound. When data on at least one feature extracted from a certain sound signal generated from the body is input, the abnormal lung sound analysis model according to an embodiment of the present inventive concepts may be a learning model that outputs a result regarding whether abnormal lung sound is present in the sound signal (e.g., which abnormal lung sound is present with which degree of probability); whether a respiratory disease is present (for example, which respiratory disease is present with which degree of probability); and whether the condition of abnormal lung sound or respiratory disease is improving or worsening (for example, to which extent the condition is improving or deteriorating). For example, these results may be output in various forms, such as values related to at least one of probability, a vector, a matrix, and a coordinate. For example, the learning model may be configured with a machine learning algorithm, such as logistic regression, support vector machine (SVM), artificial neural networks (ANN), random forest, and the like. Meanwhile, the learning model according to the present inventive concepts is not necessarily limited to those listed above, but variously changed within the range that can achieve the object of the present inventive concepts.

In addition, types of abnormal lung sound according to an embodiment of the present inventive concepts may include wheezing, crackle, stridor, rhonchi, pleural friction rub, a mediastinal crunch, and the like.; and types of respiratory diseases include asthma, chronic obstructive pulmonary disease (COPD), bronchitis, pneumonia, Bordetella pertussis, epiglotitis, pulmonary fibrosis, and the like. In addition, such respiratory diseases may be associated with abnormal lung sound. For example, asthma is associated with wheezing; chronic obstructive pulmonary disease (COPD) is associated with wheezing, crackle, and rhonchi; bronchitis is associated with wheezing, crackle, rhonchi and mediastinal crunch; pneumonia is associated with wheezing, crackle, pleural friction rub, and mediastinal crunch; Bordetella pertussis is associated with whooping; and epiglottitis is associated with stridor, respectively.

Meanwhile, it should be noted that the types of abnormal lung sound and the types of respiratory diseases according to the present inventive concepts are not necessarily limited to those listed above, but any changes may be changed as much as possible within the scope that can achieve the object of the present inventive concepts. In addition, the types of respiratory diseases according to the present inventive concepts are not limited to those listed above, and may be further specifically subdivided. For example, in the case of pulmonary fibrosis, it may be specifically subdivided into idiopathic pulmonary fibrosis (IPF), and in the case of pneumonia, it may be subdivided into chronic hypersensitivity pneumonia (CHP), non-specific interstitial pneumonia (NSIP), and the like.

For example, the learning management unit 240 may learn the abnormal lung sound analysis model by using, as learning data, data on features of time series data, Fast Fourier Transform (FFT) spectrum (or an envelope associated therewith), a zero crossing rate, and an effective value of at least one base signal associated with the lung sound, and labeling data for the data on the features (for example, information on the presence or absence of normality, abnormal lung sound, disease name, etc.; such labeling is capable of being performed in advance by medical professionals). For example, the model may be configured with an input layer, at least one hidden layer, and an output layer.

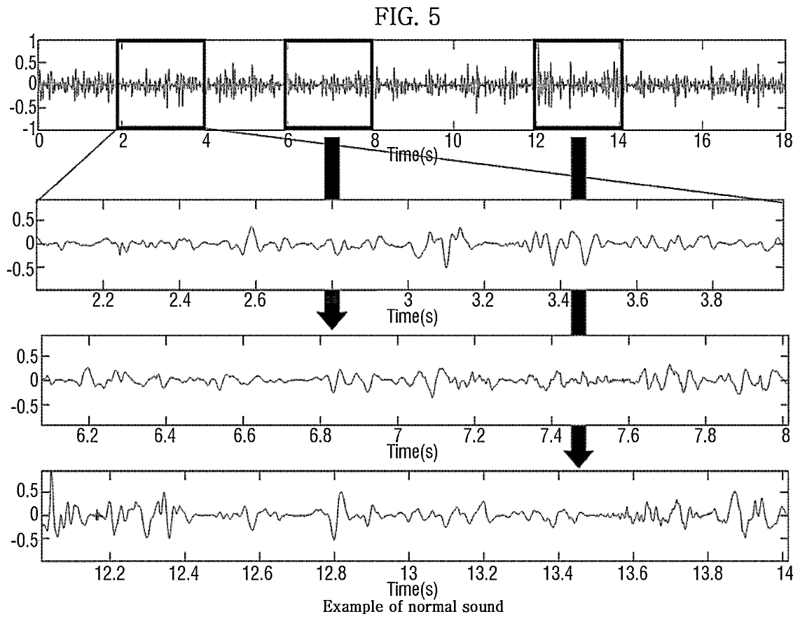

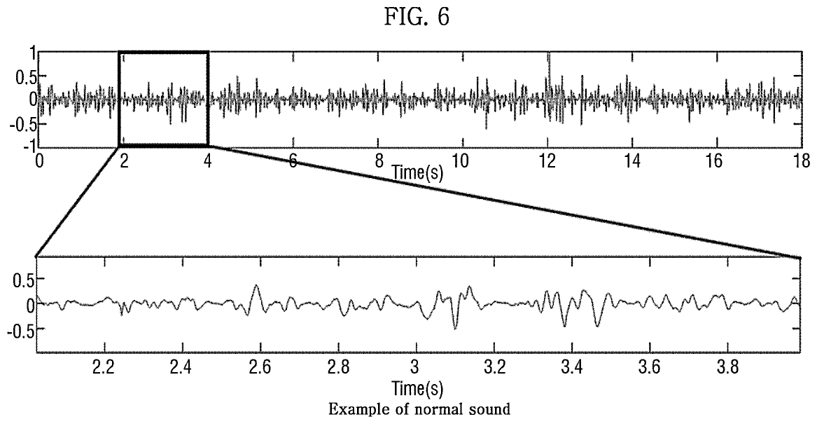

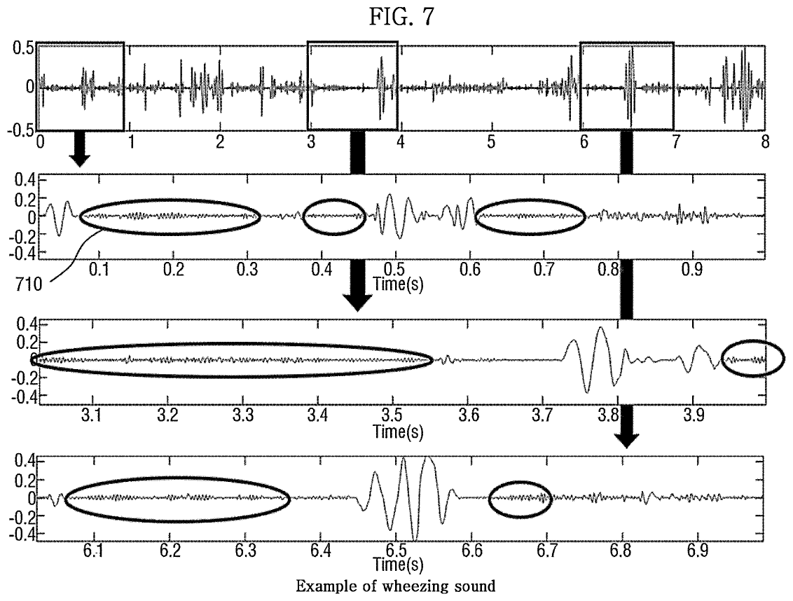

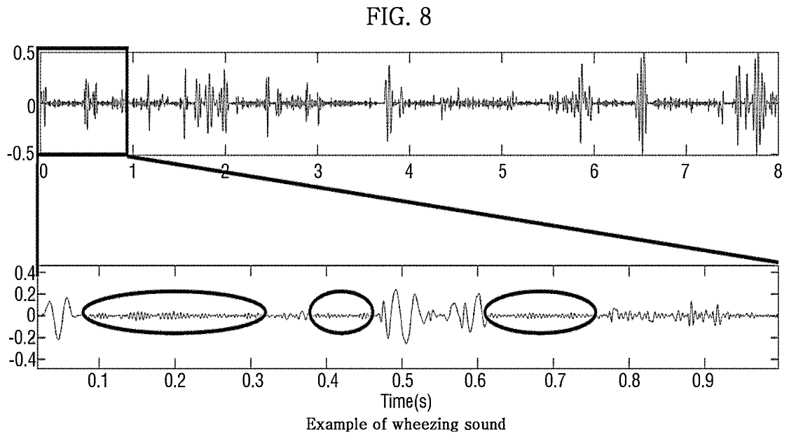

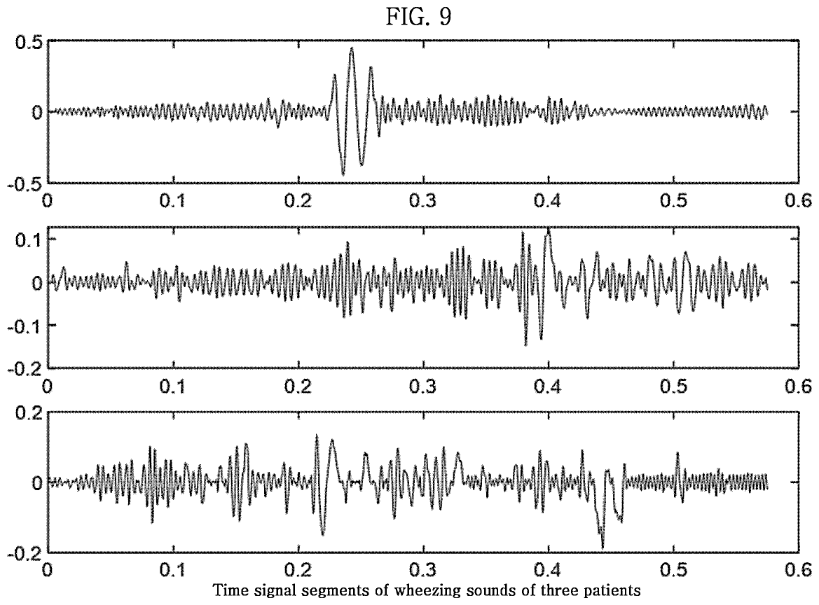

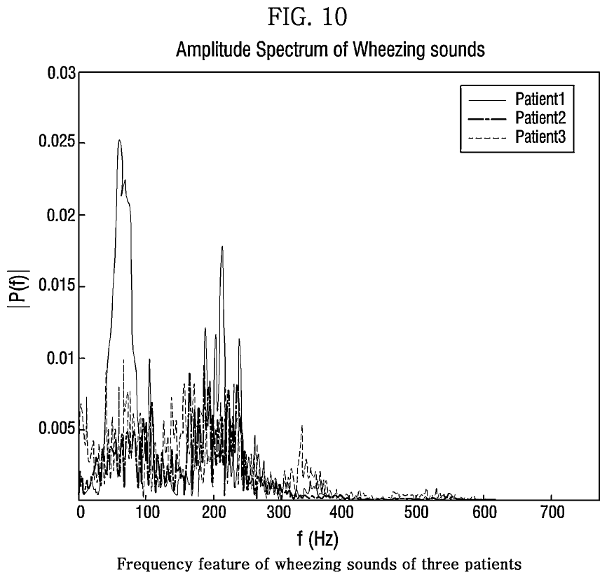

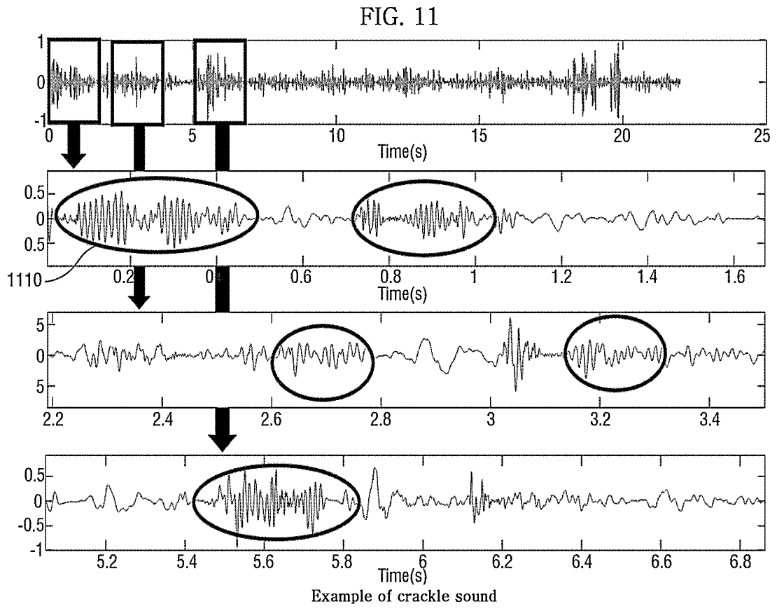

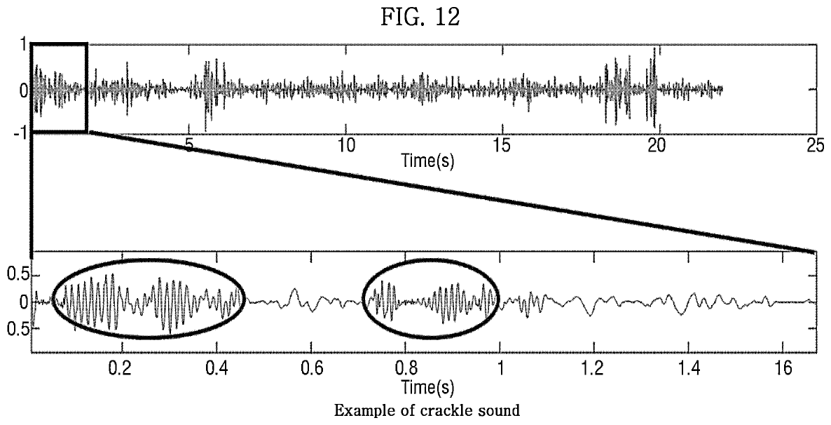

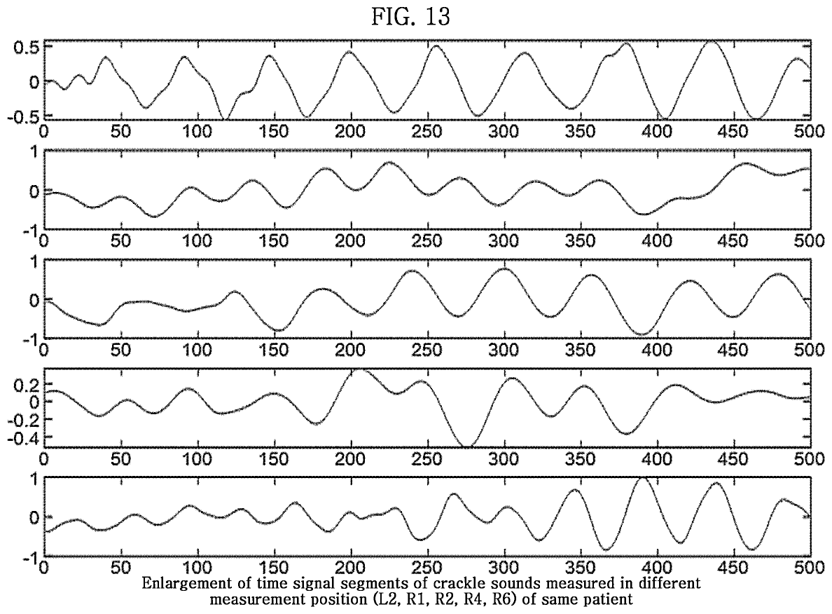

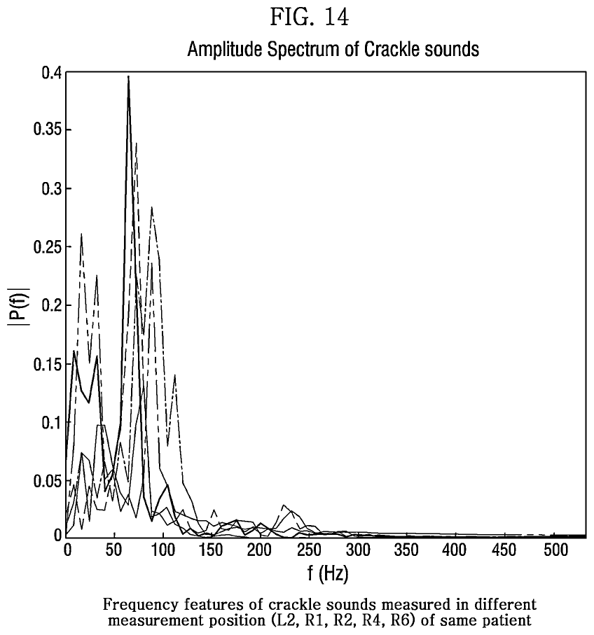

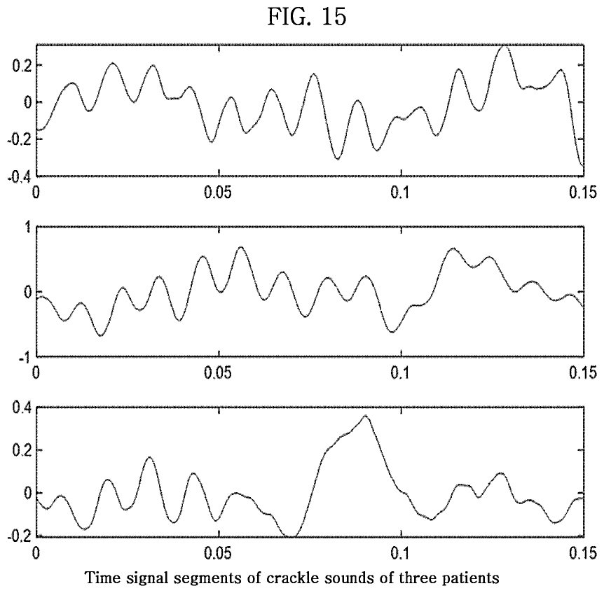

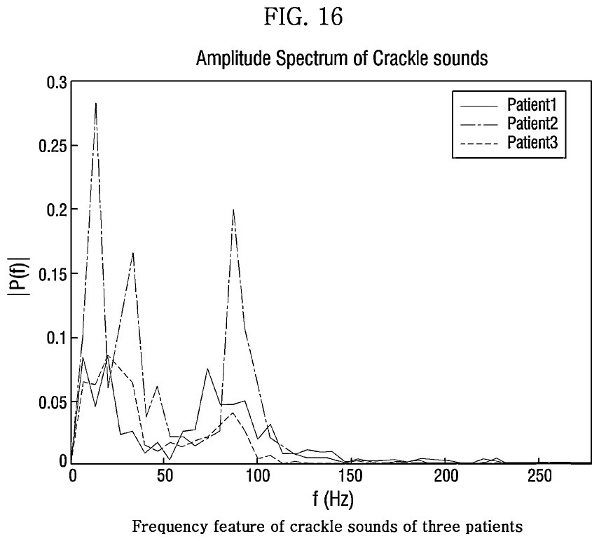

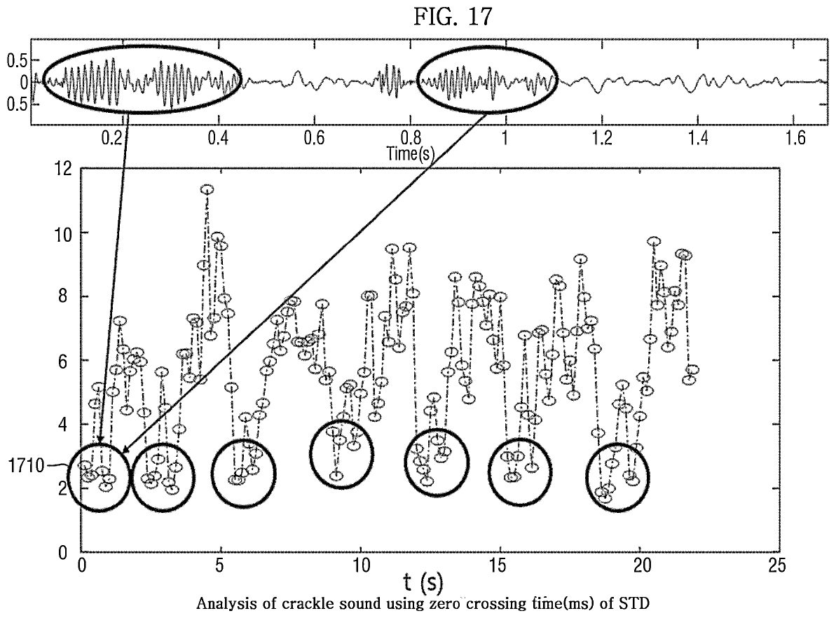

More specifically, FIGS. 5 to 17 according to an embodiment of the present inventive concepts are diagrams exemplarily showing features for each symptom specified by analyzing the base signal associated with the lung sound on the basis of a normal sound, a wheezing sound, and a crackle sound obtained from the body. First, in the case of a normal sound, the base signal associated with the lung sound appears irregularly without a separate cycle or specific pattern (see FIGS. 5 and 6). Meanwhile, in the case of a wheezing sound, the base signal is modulated with the first pattern 710 having a specific frequency and amplitude in the time domain (see FIGS. 7 and 8). When analyzing the frequency feature on the basis of time signal segments of wheezing sounds of three patients (see FIG. 9), as shown in FIG. 10, the amplitude in the time domain appears rather small, but contains a large number of high-frequency components (for example, 100 to 400 Hz). In particular, peaks appear at 0 to 150 Hz and 150 to 300 Hz, respectively. In addition, in the case of a crackle sound, the base signal is modulated in a second pattern 1110 having a specific frequency and amplitude in the time domain (see FIGS. 11 and 12). As shown in FIGS. 14 and 16, when analyzing the frequency features on the basis of the time domain segments of the crackle sound measured at multiple body locations of a specific patient (see FIG. 13) and the time domain segments of the crackle sounds of three patients (see FIG. 15), peaks appear at 0 to 50 Hz and 50 to 150 Hz, respectively. Meanwhile, the peak appears in the same frequency region irrespective of the measured positions. The learning management unit 240 according to an embodiment of the present inventive concepts may extract data on features such as zero crossing rate and time series data (e.g., time domain, frequency domain) of the base signal at a time point associated with each of the above symptoms from the base signal associated with the lung sound, and enables the abnormal lung sound analysis model to be learned to detect abnormal lung sounds on the basis of the data on the feature and the labeling data thereon. In addition, the learning management unit 240 according to an embodiment of the present inventive concepts extracts data on features such as zero crossing rate and time series data (e.g., time domain, frequency domain) of a base signal associated with lung sound of a patient with a specific respiratory disease, and enables an abnormal lung sound analysis model to be learned to diagnose respiratory diseases on the basis of the data on the feature and the labeling data thereon (or on the basis of data on which abnormal lung sound is detected to which degree in the abnormal lung sound analysis model to detect the abnormal lung sound and the labeling data thereon; referring to the association between respiratory disease and abnormal lung sound discussed earlier). In addition, the learning management unit 240 according to an embodiment of the present inventive concepts may extract data on features such as frequency at which abnormal lung sound (or respiratory disease) is detected from the base signal associated with the lung sound for a predetermined period, duration of abnormal lung sound, length of abnormal lung sound compared to breathing sound, and the like, and enables the abnormal lung sound analysis model to be learned to predict disease conditions on the basis of the feature data and the labeling data thereon. Meanwhile, the present inventive concepts does not necessarily limit the types of features extracted from the base signal associated with lung sound to those listed above, but may be changed as much as possible within the range that can achieve the object of the present inventive concepts. For example, according to an embodiment of the present inventive concepts, the abnormal lung sound analysis model may be learned by using, as learning data, data on secondary features analyzed on the basis of the above features. More specifically, referring to FIG. 17, the abnormal lung sound analysis model may be learned by using, as learning data, the standard deviation (STD) specified on the basis of the time interval of the zero crossing rate within the window while moving a time window of a fixed length (for example, 0.25 seconds). Here, when the standard deviation is lower than a predetermined threshold (1710), the abnormal lung sound may be specified as a crackle sound.

Meanwhile, according to an embodiment of the present inventive concepts, the learning management unit 240 may use, as learning data, data on at least one feature extracted from the base signal associated with the lung sound to be sampled at a predetermined length longer than the respiratory cycle.

For example, the learning management unit 240 performs sampling for data on at least one feature specified from the base signal associated with the lung sound in the case of data with a period (e.g., 8 seconds) longer than the respiratory cycle, and performs pre-processing to match the period of other data through zero-padding in the case of data with shorter period.

In addition, the learning management unit 240 according to an embodiment of the present inventive concepts may learn the abnormal lung sound analysis model by further using, as learning data, data on at least one feature specified from a base signal other than the base signal associated with the lung sound, from among the plurality of base signals.

For example, referring back to FIG. 3, the learning management unit 240 may perform learning on the abnormal lung sound analysis model by further using, as learning data, at least one of time series data, a zero crossing rate, and an effective value of the base signal 340 associated with the heart, whereby it is possible to further analyze heart health or to analyze respiratory diseases associated with heart sounds.

Next, the analysis management unit 250 according to an embodiment of the present inventive concepts may perform at least one of abnormal lung sound detection, respiratory disease diagnosis, and disease condition prediction by using the abnormal lung sound analysis model, when the learning is completed by the learning management unit 240.

For example, the analysis management unit 250 performs two or more of abnormal lung sound detection, respiratory disease diagnosis, and disease condition prediction by using one abnormal lung sound analysis model, or performs abnormal lung sound detection, respiratory disease diagnosis, and disease condition prediction by using each abnormal lung sound analysis model. Here, the abnormal lung sound analysis model may include an abnormal lung sound analysis model for detecting abnormal lung sounds, an abnormal lung sound analysis model for diagnosing respiratory diseases, and an abnormal lung sound analysis model for predicting disease conditions.

Next, the communication unit 260 according to an embodiment of the present inventive concepts, as shown in FIG. 2, may perform a function of enabling data transmission and reception to/from the sound signal acquisition unit 210, the filtering management unit 220, the base signal determination unit 230, the learning management unit 240, and the analysis management unit 250.

Finally, the control unit 270 according to an embodiment of the present inventive concepts may perform a function of controlling the data flow between the sound signal acquisition unit 210, the filtering management unit 220, the base signal determination unit 230, the learning management unit 240, the analysis management unit 250, and the communication unit 260. That is, the control unit 270 according to the present inventive concepts controls the data flow to/from the outside of the sound analyzing system 200 or the data flow between components of the sound analyzing system 200, thereby performing unique functions in each of the sound signal acquisition unit 210, the filtering management unit 220, the base signal determination unit 230, the learning management unit 240, the analysis management unit 250, and the communication unit 260.

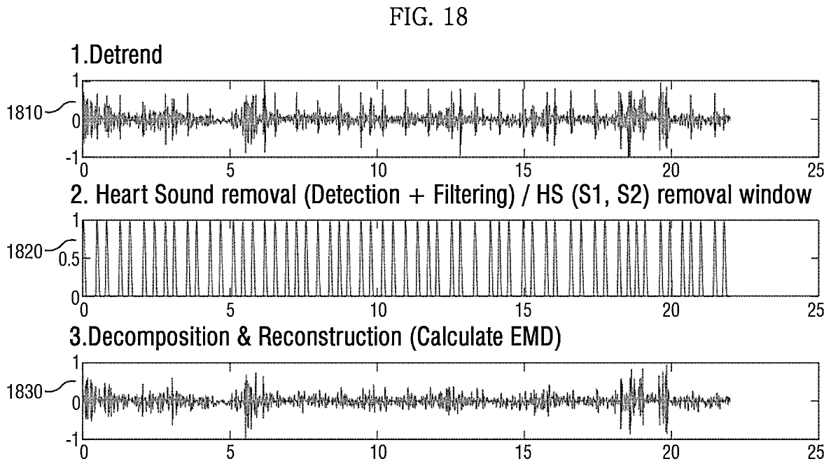

Next, referring to FIG. 18, it represents a diagram exemplarily illustrating a process of analyzing a breathing-related sound according to an embodiment of the present inventive concepts.

Referring to FIG. 18, according to an embodiment of the present inventive concepts, it may be assumed that the patient's smartphone 300 includes the sound analyzing system 200 according to the present inventive concepts.

First, according to an embodiment of the present inventive concepts, sound signals generated from the patient's body may be directly acquired in real time through the patient's smartphone 300, or may be acquired from at least one pre-stored recording device (e.g., server, cloud, etc.).

Then, the patient's smartphone 300 according to an embodiment of the present inventive concepts may filter out linear components (e.g., a DC component) of a sound signal on the basis of a predetermined detrend algorithm (1810), and filter out noises including the heart sound from the detrend sound signal (1820).

Then, the patient's smartphone 300 according to an embodiment of the present inventive concepts decomposes the filtered sound signal into a plurality of base signals having different frequencies on the basis of the empirical mode decomposition, and determines a base signal associated with lung sound from among the plurality of base signals (1830).

For example, when the filtered sound signal is decomposed into ten base signals having different frequencies (specifically when the sound signal is decomposed into ten base signals and residual signals), a specific base signal associated with the lung sound may be selected from among the ten base signals. More specifically, ten base signals are configured such that, in the order of higher frequency, the first base signal may be the base signal related to the background noise, the second to fourth base signals may be base signals associated with the lung sound, the fifth and sixth base signals may be base signals associated with the heart sound, the seventh to tenth base signal may be base signals associated with other noises (i.e., other than background noise, lung sound, and heart sound). Here, the second to fourth base signals may be determined as the base signal associated with the lung sound.

Then, according to an embodiment of the present inventive concepts, the patient's smartphone 300 may enable the abnormal lung sound analysis model to be learned using, as learning data, data on at least one feature specified from the base signal associated with the lung sound. Meanwhile, the patient's smartphone 300 may use a cloud or an external server to reduce computational loads generated in the above learning process.

For example, the patient's smartphone 300 extracts data on features such as its time-series waveform, frequency spectrum, mel-frequency cepstral coefficients (MFCC), linear frequency cepstral coefficients (LFCC), zero crossing rate, formant frequency, log energy (log E), kurtosis, and spectral centroid, from the base signal associated with lung sound, and enables an abnormal lung sound analysis model to be learned using, as learning data, the data on the feature and the labeling data on the data on the feature. Here, the abnormal lung sound analysis model may include any one of an abnormal lung sound analysis model for detecting abnormal lung sound, an abnormal lung sound analysis model for diagnosing a respiratory disease, and an abnormal lung sound analysis model for predicting a condition.

Then, when learning is completed for the abnormal lung sound analysis model according to an embodiment of the present inventive concepts, the patient's smartphone 300 may perform at least one of abnormal lung sound detection, respiratory disease diagnosis, and disease condition prediction by using the above abnormal lung sound analysis model.

Although it has been described with respect to an embodiment that enables the abnormal lung sound analysis model to be learned using data on the features specified from the base signal associated with the lung sound, and performs abnormal lung sound detection, respiratory disease diagnosis, disease condition prediction, etc., on the basis of the learning, it is noted that the present inventive concepts is not necessarily limited to using the learned model, but can be expanded, within the range that can achieve the objective of the present inventive concepts, into a manner using pattern matching, for example, a manner of detecting abnormal lung sound, diagnosing respiratory diseases, predicting disease conditions, etc., by comparing the data on the features specified from the base signal associated with the patient's lung sound with the data on the features recorded in the lookup table or database. Here, data on features and data on symptoms, diseases, etc. labeled with respect to the feature data may be recorded in the database.

The above-described embodiments according to the present inventive concepts may be implemented in the form of program instructions that can be executed through various computer components and recorded in a non-transitory computer-readable recording medium. The non-transitory computer-readable recording medium may include program instructions, data files, data structures, etc. alone or in combination. The program instructions recorded on the non-transitory computer-readable recording medium are specially designed and configured for the purpose of the present inventive concepts, or may be known and available to those skilled in the art of computer software. Examples of non-transitory computer-readable recording media include magnetic media such as hard disks, floppy disks and magnetic tape; optical recording media such as CD-ROM and DVD; magneto-optical media, such as floptical disks; and a hardware device specially configured to store and execute program instructions, such as ROM, RAM, flash memory, etc. Examples of program instructions include not only machine language codes generated by a compiler, but also high-level language codes capable of being executed by a computer using an interpreter or the like. The hardware device may be configured to operate as one or more software modules for carrying out the processing according to the present inventive concepts, and vice versa.

Although the present inventive concepts has been described with specific matters such as specific components and limited embodiments and drawings in the foregoing, this is only provided to help a more general understanding of the present inventive concepts. Therefore, it should be noted that the present inventive concepts is not limited to the above embodiments, and those skilled in the art to which the present inventive concepts pertains can make various modifications and variations from these descriptions.

Therefore, the spirit of the present inventive concepts should not be limited to the above-described embodiment, and all modifications equivalently or equivalent to these claims as well as the claims described below shall fall within the scope of the spirit of the present inventive concepts.

1-13. (canceled)

14. A method of analyzing a breathing-related sound, comprising:
receiving a sound signal obtained while a recording device is coupled to a body;
filtering noises from the sound signal, the noises comprising at least a signal related to a sound from a heart of the body;
decomposing the filtered sound signal into a plurality of base signals based on empirical mode decomposition, wherein a first base signal of the plurality of base signals has different frequencies from a second base signal of the plurality of base signals;
determining a base signal, associated with a sound from a lung of the body, from the plurality of base signals; and
obtaining information relating to the lung of the body using a sound analysis model and the determined base signal, wherein the sound analysis model is trained with a training base signal and labeling data indicating an abnormality of the sound from the lung of the body, wherein a criterion for determining the training base signal is the same as a criterion at the determining step to determine the base signal from among a plurality of base signals.

15. The method of claim 14, wherein the determining comprises determining the base signal associated with the sound from the lung and comparing with a reference signal based on a similarity of amplitude, characteristics of frequency, and pattern of a wave.

16. The method of claim 14, wherein the determining comprises:
arranging the plurality of basis signals according to an order of frequency magnitudes; and
determining the base signal arranged at a preset order as the base signal associated with the sound from the lung.

17. The method of claim 14, wherein the information relating to the lung indicates at least one of wheezing, crackle, stridor, rhonchi, pleural friction rub, or mediastinal crunch.

18. The method of claim 14, wherein the information relating to the lung indicates at least one of asthma, COPD, bronchitis, pneumonia, Bordetella pertussis, or epiglottitis.

19. The method of claim 14, wherein the obtaining comprises generating input data based on the determined base signal, wherein a form of the input data is time series data, Fast Fourier Transform (FFT) spectrum, a zero crossing rate, or an effective value.

20. The method of claim 14, wherein the filtering is performed by filtering out the noises from the sound signal by using a bandpass filter,
wherein the bandpass filter is set to filter 20 Hz-300 Hz on the basis of features of the sound from the heart.

21. A non-transitory computer-readable recording medium comprising computer readable instructions stored thereon that, when executed by at least one process, configure the at least one processor to,
receive a sound signal obtained while a recording device is coupled to a body;
filter noises from the sound signal, the noises comprising at least a signal related to a sound from a heart of the body;
decompose the filtered sound signal into a plurality of base signals based on empirical mode decomposition, wherein a first base signal of the plurality of base signals has different frequencies from a second base signal of the plurality of base signals;
determine a base signal, associated with a sound from a lung of the body, from the plurality of base signals; and
obtain information relating to the lung of the body using a sound analysis model and the determined base signal, wherein the sound analysis model is trained with a training base signal and labeling data indicating an abnormality of the sound from the lung of the body, wherein a criterion for determining the training base signal is the same as a criterion at the determining step to determine the base signal from among a plurality of base signals

22. A system for analyzing a breathing-related sound, comprising:
a sound signal acquisition unit configured to acquire a sound signal generated from the body;
a filtering management unit configured to filter noises from the sound signal, the noises comprising at least a signal related to a sound from a heart of the body;
a base signal determination unit configured to decompose the filtered sound signal into a plurality of base signals based on empirical mode decomposition, wherein a first base signal of the plurality of base signals has different frequencies from a second base signal of the plurality of base signals; and
determining a base signal, associated with a sound from a lung of the body, from the plurality of base signals; and
a learning management unit configured to obtain information related to the lung of the body using a sound analysis model and the determined base signal, wherein the sound analysis model is trained with a training base signal and labeling data indicating an abnormality of the sound from the lung of the body, wherein a criterion for determining the training base signal and the base signal associated with the sound of the lung of the body

23. The system of claim 22, wherein the base signal determination unit determines the base signal associated with the sound from the lung compared with a reference signal based on a similarity of amplitude, characteristics of frequency, and pattern of a wave.

24. The system of claim 22, wherein the base signal determination unit arranges the plurality of basis signals according to an order of frequency magnitudes, and determines the base signal arranged at a preset order as the base signal associated with the sound from the lung.

25. The system of claim 22, wherein the information relating to the lung indicates at least one of wheezing, crackle, stridor, rhonchi, pleural friction rub, or mediastinal crunch.

26. The system of claim 22, wherein the information relating to the lung indicates at least one of asthma, COPD, bronchitis, pneumonia, Bordetella pertussis, or epiglottitis.

27. The system of claim 22, wherein the learning management unit is configured to generate input data based on the determined base signal and obtains information relating to the lung, and
wherein a form of the input data is time series data, Fast Fourier Transform (FFT) spectrum, a zero crossing rate, or an effective value.

28. The system of claim 22, wherein the filtering management unit filters out the noises from the sound signal by using a bandpass filter,
wherein the bandpass filter is set to filter 20 Hz-300 Hz on the basis of features of the sound from the heart.

29. The non-transitory computer readable medium of claim 21, wherein the at least one processor is further configured to execute the computer readable instructions to,
determine the base signal associated with the sound from the lung and comparing with a reference signal based on a similarity of amplitude, characteristics of frequency, and pattern of a wave.

30. The non-transitory computer readable medium of claim 21, wherein the at least one processor is further configured to execute the computer readable instructions to,
determine the base signal arranged at a preset order as the base signal associated with the sound from the lung.

31. The non-transitory computer readable medium of claim 21, wherein the information relating to the lung indicates at least one of wheezing, crackle, stridor, rhonchi, pleural friction rub, mediastinal crunch, asthma, COPD, bronchitis, pneumonia, Bordetella pertussis, or epiglottitis.

32. The non-transitory computer readable medium of claim 21, wherein the at least one processor is further configured to execute the computer readable instructions to,
generate input data based on the determined base signal and obtains information relating to the lung, and wherein a form of the input data is time series data, Fast Fourier Transform (FFT) spectrum, a zero crossing rate, or an effective value.

33. The non-transitory computer readable medium of claim 21, wherein the filtering is performed by filtering out the noises from the sound signal by using a bandpass filter, wherein the bandpass filter is set to filter 20 Hz-300 Hz on the basis of features of the sound from the heart.
